(19) United States
(12) Patent Application Publication
(10) Pub. No.: US 2012/0031413 A1
(43) Pub. Date: Feb. 9, 2012

(54) MASK

(75) Inventors: ZHENG SHI, Shenzhen City (CN); JIANG-FENG LIU, Shenzhen City (CN); YU-XI GAO, Shenzhen City (CN)

(73) Assignees: FIH (HONG KONG) LIMITED, Kowloon (HK); SHENZHEN FUTAIHONG PRECISION INDUSTRY CO., LTD., ShenZhen City (CN)

(21) Appl. No.: 12/911,854

(22) Filed: Oct. 26, 2010

(30) Foreign Application Priority Data
Aug. 6, 2010 (CN) 201010247295.4

(51) Int. Cl. A61F 9/04; A42B 1/18

(52) U.S. Cl. 128/858; 2/173; 2/206

(57) ABSTRACT

A mask includes a support member, a transparent main body, and a fixing member. The support member includes a main portion, a holding portion, and a resisting portion. The holding portion is integrally formed with the main portion. The holding portion extends away from the transparent main body. The resisting portion is positioned on the main portion. The transparent main body is fixed on the main portion. The fixing member is connected to the main portion.

### CROSS-REFERENCE TO RELATED APPLICATIONS

This application is one of the three related co-pending U.S. patent applications listed below. All listed applications have the same assignee and were concurrently filed herewith. The disclosure of each of the listed applications is incorporated by reference into all the other listed applications.

| Attorney Docket No. | Title | Inventors |
|---|---|---|
| US34789 | MASK | Shi et al. |
| US34790 | MASK | Shi et al. |
| US34798 | MASK | Shi et al. |

Furthermore, this application is related to the following design patent filed on Aug. 19, 2010.

| Attorney Docket No. | Title | Inventors |
|---|---|---|
| US34791 | MASK | Shi et al. |

### BACKGROUND

1. Technical Field

The present disclosure generally relates to masks, and particularly, to a transparent mask.

2. Description of Related Art

It is well known that masks are widely applied in medical, health, catering services, and etc. to restrict contamination. A mask often includes a main body and fixing members respectively connecting opposite ends of the main body. The main body often includes layers of gauze and contains material exhaled or expelled by the wearer. The fixing members can attach to the user's ears. However, since the mask completely covers the face and nose, with time discomfort may be experienced. Furthermore, the mask hides facial expressions, limiting communication with other people.

Therefore, there is a room for improvement within the art.

### BRIEF DESCRIPTION OF THE DRAWINGS

Many aspects of the mask can be better understood with reference to the following drawings. The components in the drawings are not necessarily to scale, the emphasis instead being placed upon clearly illustrating the mask. Moreover, in the drawings, like reference numerals designate corresponding parts throughout the several views.

FIG. 1 is an isometric view of a mask, according to an exemplary embodiment.

FIG. 2 is an isometric view of the mask shown in FIG. 1, from another angle.

FIG. 3 is an isometric view of the mask shown in FIG. 2, from another angle.

FIG. 4 is an exploded view of the mask shown in FIG. 1.

### DETAILED DESCRIPTION

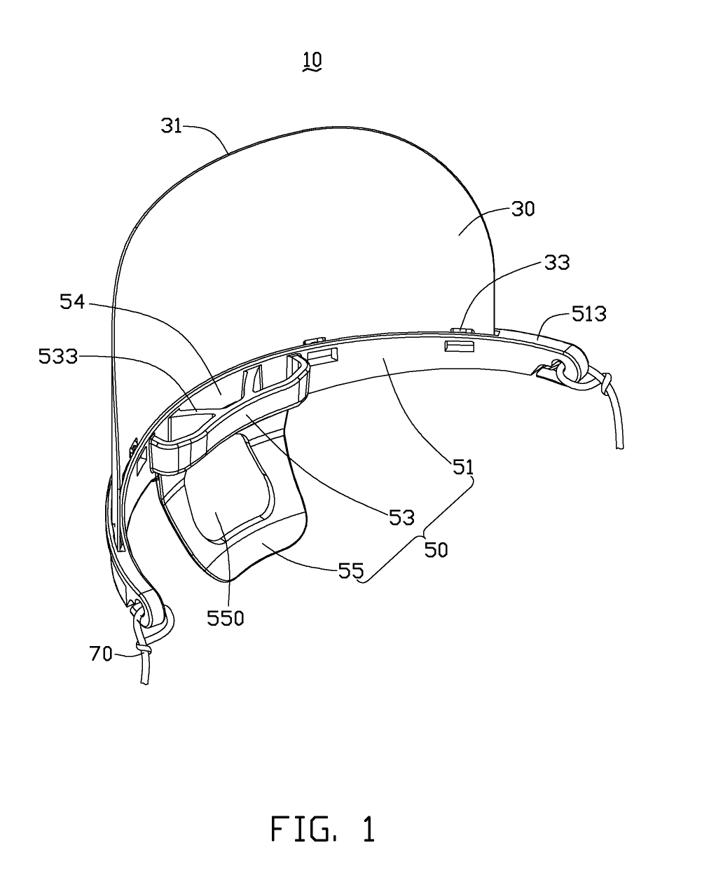

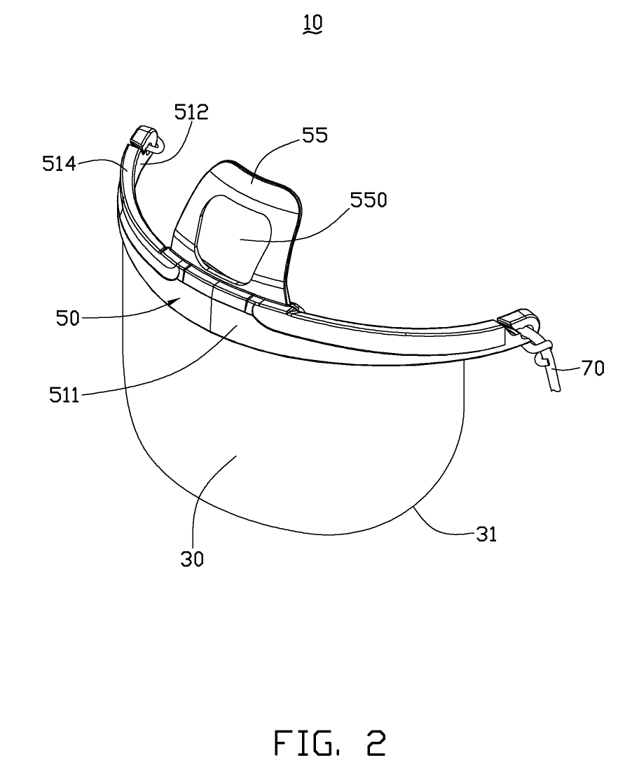

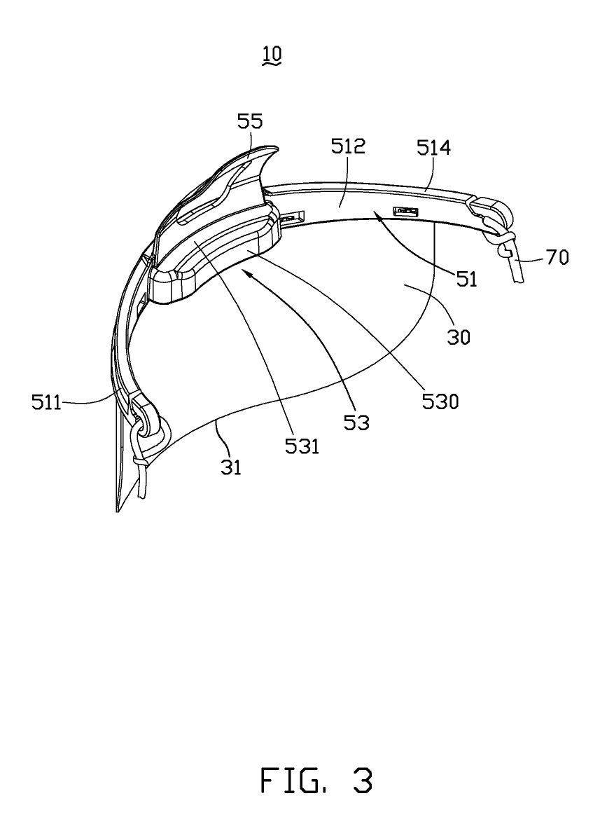

FIG. 1 to FIG. 3 show an exemplary embodiment mask including a transparent main body 30, a support member 50 connected to the transparent main body 30, and a fixing member 70 fixed on the support member 50.

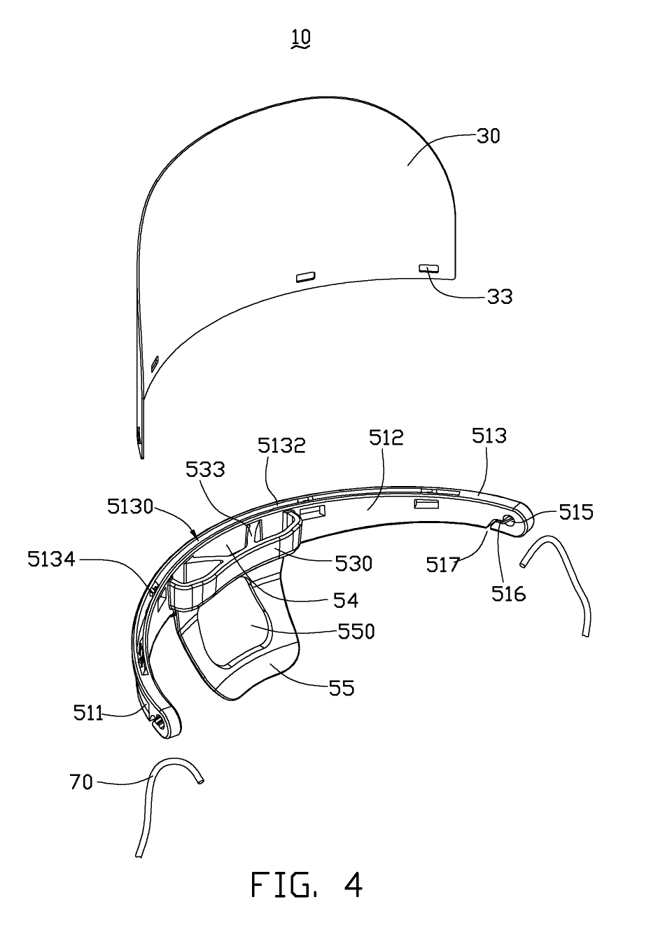

Referring to FIG. 4, the transparent main body 30 may be made of polycarbonate (PC), and is a curved sheet for covering a user's nose and part of the face. The transparent main body 30 includes a curved edge 31 and a plurality of latching holes 33 defined in an opposite end to the curved edge 31.

The support member 50 includes a main portion 51, a resisting portion 53, and a holding portion 55 integrally formed together. The main portion 51 may be in the shape of a bar and made of acrylonitrile-butadiene-styrene (ABS). The main portion 51 includes a convex surface 511, a concave surface 512, a first surface 513, and a second surface 514 opposite to the first surface 513. The first surface 513 and the second surface 514 connect the convex surface 511 to the concave surface 512. The first surface 513 defines a receiving slot 5130 for receiving the end of the main body 30 defining the latching holes 33. A plurality of blocks 5134 is formed on an inner wall 5132 of the receiving slot 5130. In this exemplary embodiment, the blocks 5134 extend from the inner wall 5132 to the direction of concave surface 512. The blocks 5134 are configured for matching the latching holes 33, thus, the transparent main body 30 can be fixed on the support member 50. Ends of the main portion 51 respectively define a through hole 515 and a guiding slot 516. The through hole 515 and the guiding slot 516 communicate the convex surface 511 with the concave surface 512. The guiding slot 516 extends through the second surface 514 to the through hole 515 to form an entrance 517. The fixing member 70 can pass through the entrance 517 and be received in the through hole 515 for tying the main body 30.

The resisting portion 53 is positioned on a middle of the concave surface 512. The resisting portion 53 includes a frame 530, a bottom plate 531, and ribs 533. The frame 530 is curved, and free ends of the frame 530 are connected to the concave surface 512. The bottom plate 531 connects the frame 530 to the concave surface 512. Thus, the bottom plate 531, the frame 530, and the concave surface 512 cooperatively define a receiving chamber 54. The ribs 533 are formed in the receiving chamber 54 for connecting the frame 530 to the concave surface 512. The resisting portion 53 contacts the user between the lower jaw and the lip, defining between the user's face and the support member 50, such that the user's nose is not covered.

The holding portion 55 is positioned on the second surface 514, extending away from the transparent main body 30, and following the concave surface 512. The holding portion 55 defines an opening 550. The holding portion 55 fixes the user's lower jaw to prevent movement when the mask 10 is worn.

The fixing member 70 can enter the entrance 517 and tie in the through hole 515, thereafter assembled to the support member 50.

In assembly, the end the transparent main body 30 defining the latching holes 33 is received in the receiving slot 5130. The blocks 5134 are received in the corresponding latching holes 33, and the receiving chamber 5138 is received in the notch 35. Thus, the transparent main body 30 is fixed on the support member 50. Ends of the fixing member 70 pass through the entrance 517 and are tied in the through hole 515. Thus, the fixing member 70 is tied to the support member 50.

When the mask 10 is worn, the resisting portion 53 contacts the concave surface between the lower jaw and the lip of the user, and the holding portion 55 fixes the user's lower jaw. The fixing member 70 is fixed to the user's head. Thus, the transparent main body 30 partially masks the user's face.

The mask 10 keeps a certain distance from the user's nose, avoiding cramped sensation. In addition, the mask 10 has a transparent main body 30, such that the user's facial expression can be seen.

In another embodiment, fixing members 70 can be respectively tied to ends of the support member 50 and fixed to the user's ears.

In another embodiment, the guiding slot 516 and the entrance 517 can be omitted and fixing member 70 tied in the through hole 515 directly.

The receiving chamber 54 also can receive matter exhaled or expelled by the user.

It is to be understood, however, that even through numerous characteristics and advantages of the present disclosure have been set forth in the foregoing description, together with details of the structure and function of the disclosure, the disclosure is illustrative only, and changes may be made in detail, especially in matters of shape, size, and arrangement of parts within the principles of the disclosure to the full extent indicated by the broad general meaning of the terms in which the appended claims are expressed.

What is claimed is:

1. A mask for protecting a face of a user, comprising:
a transparent main body for positioning in front of a user's face;
a support member comprising:
a main portion fixed on the transparent main body; and
a curved holding portion extending away from the main portion for frictionally contacting a portion of the mask to the user's lower jaw to prevent the mask from moving.

2. The mask as claimed in claim 1, further comprising a resisting portion wherein, the resisting portion is positioned on the main portion.

3. The mask as claimed in claim 2, wherein the resisting portion comprises a frame, free ends of the frame connect the main portion.

4. The mask as claimed in claim 3, wherein the resisting portion comprises a bottom plate; the bottom plate connects the frame to the main portion to cooperatively define a receiving chamber.

5. The mask as claimed in claim 4, wherein the resisting portion comprises ribs formed in the receiving chamber, the ribs connect the frame to the main portion.

6. The mask as claimed in claim 2, wherein the main portion comprises a convex surface, a concave surface, a first surface, and a second surface opposite to the first surface; the transparent main body is fixed on the first surface; the holding portion is positioned on the second surface; the resisting portion is positioned on the concave surface.

7. The mask as claimed in claim 6, wherein the transparent main body defines a plurality of latching holes, the first surface defines a receiving slot, and a plurality of blocks is formed in the receiving slot, the latching holes match with the blocks.

8. The mask as claimed in claim 1, wherein ends of the main portion define a through hole for connecting a fixing member.

9. The mask as claimed in claim 8, wherein the main portion further defines guiding slots at ends thereof, each guiding slot communicates with the through hole to form an entrance.

10. A mask, comprising:
a transparent main body;
a support member comprising:
a main portion secured to the transparent main body; and
a holding portion integrally formed with the main portion for preventing the mask from moving; and
a resisting portion positioned on the main portion for resisting one portion of a user between a lower jaw and a lip.

11. The mask as claimed in claim 10, wherein the resisting portion comprises a frame, free ends of the frame connect the main portion.

12. The mask as claimed in claim 11, wherein the resisting portion comprises a bottom plate; the bottom plate connects the frame to the main portion to cooperatively define a receiving chamber.

13. The mask as claimed in claim 12, wherein the resisting portion comprises ribs formed in the receiving chamber, the ribs connect the frame to the main portion.

14. The mask as claimed in claim 10, wherein the main portion comprises a convex surface, a concave surface, a first surface, and a second surface opposite to the first surface; the transparent main body is fixed on the first surface; the holding portion is positioned on the second surface; the resisting portion is positioned on the concave surface.

15. The mask as claimed in claim 14, wherein the transparent main body defines a plurality of latching holes, the first surface defines a receiving slot, and a plurality of blocks is formed in the receiving slot, the latching holes match with the blocks.

16. The mask as claimed in claim 10, wherein ends of the main portion define a through hole for connecting a fixing member.

17. The mask as claimed in claim 16, wherein the main portion defines guiding slots at ends thereof, each guiding slot communicates with the through to form an entrance.

18. A mask comprising:
a support member comprising:
a main portion; and
a holding portion formed with the main portion, the holding portion extending away from the main portion to hold the user's lower jaw; and
a resisting portion positioned for frictionally contacting a portion of the mask to the lower jaw and the lip of the user;
a transparent main body fixed on the main portion to partially mask the user's face; and
a fixing member connected the main portion to tie on the head of the user.

19. The mask as claimed in claim 18, wherein the resisting portion comprises a frame, a bottom plate, and ribs; the free ends of the frame connect the main portion; the bottom plate connects the frame to the main portion; the bottom plate connects the frame to the main portion to cooperatively define a receiving chamber; the ribs are formed in the receiving chamber, and ribs connect the frame to the main portion.

20. The mask as claimed in claim 18, wherein ends of the main portion define a through hole for connecting a fixing member.
